(19) United States
(12) Patent Application Publication
(10) Pub. No.: US 2017/0055815 A1
(43) Pub. Date: Mar. 2, 2017

(54) IMAGING SYSTEM, METHOD AND DISTAL ATTACHMENT FOR MULTIDIRECTIONAL FIELD OF VIEW ENDOSCOPY

(71) Applicant: The General Hospital Corporation, Boston, MA (US)

(72) Inventors: Kevin E. WOODS, Atlanta, GA (US); Brett Eigene BOUMA, Quincy, MA (US); Guillermo J. TEARNEY, Cambridge, MA (US); Dongkyun KANG, Somerville, MA (US); Tao WU, Cambridge, MA (US)

(21) Appl. No.: 15/306,581

(22) PCT Filed: Apr. 24, 2015
(86) PCT No.: PCT/US2015/027605

§ 371 Date: Oct. 25, 2016

Related U.S. Application Data
(60) Provisional application No. 61/984,258, filed on Apr. 25, 2014.

(51) Int. Cl. A61B 1/06; H04N 5/225; G02B 23/24; A61B 1/00

(52) U.S. Cl. CPC A61B 1/0638; A61B 1/00137; A61B 1/0623; A61B 1/00181; H04N 5/2256; G02B 23/2461; G02B 23/2423; H04N 2005/2255

(57) ABSTRACT

An exemplary apparatus for imaging at least one anatomical structure can be provided. For example, the apparatus can include a probe imaging first arrangement, a radiation source second arrangement which provides at least one electro-magnetic radiation, and a third arrangement attached to at least one portion of the endoscopic arrangement. The third arrangement can contain an optical arrangement which, upon impact by the at least one electro-magnetic radiation and based thereon, may transmit a first radiation and reflects a second radiation. The first radiation can impact at least one first portion of the anatomical structure(s), and the second radiation can impact at least one second portion of the anatomical structure(s). The first and second portions can be at least partially different from one another. Further, the first and second radiations can have characteristics which are different from one another. In addition, In addition, the third arrangement can include a hollow internal portion that facilitates an unobstructed passage of at least one of fluid or instrument within an accessory channel of the first arrangement.

### CROSS-REFERENCE TO RELATED APPLICATION(S)

This application claims priority from U.S. patent application Ser. No. 61/984,258, filed on Apr. 25, 2014, and also relates to International Application PCT/US2013/031948 filed Mar. 15, 2013 (and published as International Patent Publication WO 2013/148306 on Oct. 3, 2013) and U.S. patent application Ser. No. 61/618,225, filed Mar. 30, 2012, the entire disclosures of which are incorporated herein by reference.

### FIELD OF THE DISCLOSURE

Exemplary embodiments of the present disclosure relate to endoscopic imaging system and methods for multidirectional field of view endoscopy which can be used to improve the field of view, speed and efficiency of diagnostic and therapeutic endoscopic procedures.

### BACKGROUND OF THE DISCLOSURE

In general, endoscopic imaging systems allow the evaluation of animal and human internal organs. Endoscopes can consist of at least one of the following components, a rigid or flexible tube, a light delivery system, a fluid delivery and recovery system, an air delivery and recovery system, a lens system, an eyepiece, a high pixel-count color CCD or imaging transmission system, graphical display unit (monitor), and/or accessory channel(s) to allow use of devices for manipulation, sampling or imaging of target lesions.

The endoscope may be inserted into any natural orifice of the animal or human including the nares, ears, mouth, biliary tract, pancreatic duct, ostomy, urinary tract, vagina, uterus, fallopian tubes, anus and/or any opening produced by procedures employing an incision or puncture into an internal body cavity (craniotomy, thoracotomy, mediastinotomy, laparotomy or arthrotomy). While currently available endoscopes are capable of evaluating target structures by the obligatory forward or other directional field of view obtained by current light delivery and lens systems, in some medical applications this design increases the risk for missed detection of important areas of interest. As a result, there is a need for multi-directional visualization.

Colonoscopy is widely considered the gold standard for detecting mucosal abnormalities in the human colon, and the preferred technique for removal of many non-invasive lesions requires biopsy, polypectomy or endoscopic resection. There have been well-documented limitations related to the practice of colonoscopy with traditional endoscopic instruments. Because most colon cancers are believed to arise from abnormal colon tissue, adenomas, the detection and removal of adenomatous polyps have been recommended for the prevention of future colon cancers. (See, e.g., ref. 1). Missed polyps or cancers have been one of these unfortunate limitations. (See, e.g., refs. 2-4). Although there are additional factors associated with the risks of missing mucosal lesions such as, a patient's colonic anatomy, patient comfort during an endoscopic procedure and the quality of bowel preparation, it has been well established by other investigators, that the location of mucosal abnormalities is highly associated with failure of identification. (See, e.g., ref 4).

Prior groups have investigated several approaches to attempt to demonstrate an improvement in the diagnostic yield of a colonoscopic procedure by altering or increasing the conventional forward fields of view. Unfortunately these studies did not demonstrate a significant increase in adenoma detection. (See, e.g., refs. 5-7). Nevertheless, the uses of a transparent cap that does not change or improve the field of view placed on the distal aspect of colonoscopes have demonstrated great promise in improving the effectiveness of colonoscopy (see, e.g., refs. 8-11) and adenoma detection (see, e.g., ref. 12), however the use of these devices are still associated with a significant adenoma miss rate. (See, e.g., ref 13).

Other researchers have attempted to improve the adenoma detection rate established with the use of a conventional endoscopic system by increasing the total field of view during a colonoscopy by coupling the traditional endoscope with an auxiliary imaging device, placed within the accessory channel, to provide a continuous retrograde view of the target organ via the accessory channel. (See, e.g., ref. 14). While this auxiliary imaging device provides a continuous retrograde field of view used in combination with traditional forward viewing endoscopes, it requires the use of an accessory channel of the endoscope. This becomes an important factor during colonoscopy, if used with a standard single channel colonoscope, due to the necessity to remove the auxiliary imaging device to allow for the use of an appropriate auxiliary sampling or retrieval instrument to biopsy, resect and retrieve specimens removed from the organ being investigated. This additional equipment has been shown in a prospective, multicenter, randomized, controlled trial to decrease the relative risk of missing polyps and adenomas but was also shown to have a statistically significant increase in the mean total procedure times.¹⁵ Auxiliary endoscopic devices placed within the auxiliary channel of the endoscope have the further disadvantage that they require an additional endoscope, which increases complexity, ease of use, and cost of the overall procedure.

Thus, there is a need to address at least some of the issues and/or deficiencies described herein above.

### SUMMARY OF EXEMPLARY EMBODIMENTS

In various exemplary embodiments according to the present disclosure, exemplary configurations for the acquisition of multidirectional viewing during endoscopic examination can be provided. Exemplary applications can be utilized, in which increasing the field of view while using high resolution endoscopic systems can be improved with the exemplary embodiments of the system and method of continuous and simultaneous forward and multidirectional views during a baroscopic, laparoscopic, angioscopic, or endoscopic procedure.

Exemplary embodiments of the present disclosure can relate generally to exemplary configuration of optical elements, and to the application(s) thereof in exemplary endoscopic imaging systems which can be used with medical applications to improve the field of view, speed and efficiency of an endoscopic procedure. Exemplary embodiments of the present disclosure can be applied to rigid, flexible, wireless or telescoping endoscope to provide, e.g., a continuous multi-directional view of animate and inanimate hollow spaces.

In one further exemplary embodiment of the present disclosure, a distal imaging attachment and an imaging system can be used in combination with a rigid, flexible, wireless or telescoping endoscope to create a continuous multi-directional view of animate and inanimate hollow spaces. According to a further exemplary embodiment of the present disclosure, the directions are forward and to the side. In yet another preferred embodiment of the present disclosure, the directions are forward and backward. In still yet another further embodiment, the directions cover approximately a 4 pi solid angle that is only obscured by the device itself. This said distal imaging attachment and imaging system may be employed, but not limited to, with endoscopy of animal and human internal anatomical organs and borescopy of inanimate closed spaces. Due to its design, the integrated optical element within this imaging system, allowing both the forward and multidirectional fields of view.

In yet further exemplary embodiment of the present disclosure, it is also possible to accommodate the simultaneous passage of devices via the accessory channel of a video endoscope or applicable device of which the distal imaging attachment is applied. For example, optical elements in the exemplary device can be configured to facilitate a multidirectional viewing of target organs or spaces with exemplary endoscopes. In another exemplary embodiment of the present disclosure, the exemplary device can be retrofitted to alter the native conventional high definition endoscopes currently used in endoscopic procedures. In still further exemplary embodiment of the present disclosure, the exemplary device/apparatus can be disposable.

Indeed, exemplary embodiments according to the present disclosure as described herein, can be provided as exemplary endoscopic lens system(s), and can be termed as “multidirectional”, “simulview” or “retroview”, and utilized as a basis for exemplary embodiments of endoscopic systems for a deployment.

Further, an exemplary apparatus for imaging at least one anatomical structure can be provided, according to an exemplary embodiment of the present disclosure. For example, the apparatus can include a probe imaging first arrangement, a radiation source second arrangement which provides at least one electro-magnetic radiation, and a third arrangement attached to at least one portion of the endoscopic arrangement. The third arrangement can contain an optical arrangement which, upon impact by the at least one electro-magnetic radiation and based thereon, may transmit a first radiation and reflects a second radiation. The first radiation can impact at least one first portion of the anatomical structure(s), and the second radiation can impact at least one second portion of the anatomical structure(s). The first and second portions can be at least partially different from one another. Further, the first and second radiations can have characteristics which are different from one another. Additionally or alternatively, the third arrangement can include a hollow internal portion that facilitates an unobstructed passage of at least one of fluid or instrument within an accessory channel of the first arrangement.

For example, the characteristics can include or be wavelengths or polarizations. A detector arrangement can be provided, whereas the endoscopic arrangement can be associated with the radiation source arrangement and the detector arrangement. The first and second radiations can have spectral regions in red, green and blue band which do not substantially overlap with one another. The first radiation can be directed in a forward direction, and the second radiation can be directed in a backward direction or a side direction. The third arrangement can include a cap that can be connected to an end portion of the endoscopic first arrangement. In addition or alternatively, the cap can include an optical structure, which, upon impact by electro-magnetic radiation(s) and based thereon, can cause a resultant radiation to be at least side, retrograde and forward directions of the endoscopic first arrangement with respect to a viewing direction thereof.

The second radiation can simultaneously illuminate between 270 and 360 degrees of a field of view. Further, the radiation source second arrangement can include a modulation arrangement which can be configured to modulate the first and second radiations. An electronic arrangement can be provided which is configured to synchronize the second arrangement and the detector arrangement. As an alternative or in addition, the electronic arrangement can be configured to (i) synchronize the modulation arrangement and the detector arrangement, and (ii) control the detector arrangement to detect signals from the anatomical structure(s) illuminated by the first and second radiation, and separate the signals based the synchronization with the modulation arrangement.

According to further exemplary embodiments of the present disclosure, the anatomical structure(s) can be a luminal anatomical structure. The third arrangement can include at least one opening which facilitates a passage of instrumentation, air gasses and/or fluids therethrough. A tube can be provided that is associated with the third arrangement, and which provide a passage of instrumentation, air gasses and/or fluids therethrough. Further, the first and second radiations can have a specific polarization status.

Other features and advantages of the present invention will become apparent upon reading the following detailed description of exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a side cross-sectional block diagram of an imaging system/apparatus and optical elements thereof according to an exemplary embodiment of the present disclosure;

FIG. 2 is a set of view of an exemplary optical element consists a 4-faceted pyramid dichroic mirror which can transmit and reflect radiations with different characteristics according to an exemplary embodiment of the present disclosure;

FIG. 3 a block diagram of an endoscopic arrangement, a radiation source arrangement, and a detector arrangement according to exemplary embodiments of the present disclosure;

FIGS. 4(a) and 4(b) are block diagrams of exemplary modulation arrangements according to an exemplary embodiment of the present disclosure;

FIG. 5(a) is a diagram of an exemplary electronic switch based on an optical chopper according to an exemplary embodiment of the present disclosure;

FIG. 5(b) is a diagram of the exemplary electronic switch based on a first switch position according to an exemplary embodiment of the present disclosure;

FIG. 5(c) is a diagram of the exemplary electronic switch based on a second switch position according to an exemplary embodiment of the present disclosure;

FIG. 6(a) a diagram of a further exemplary electronic switch based on a galvo scanner at a first switch position according to another exemplary embodiment of the present disclosure;

FIG. 6(b) a diagram of the exemplary electronic switch of FIG. 6(a) based on the galvo scanner at a second switch position according to another exemplary embodiment of the present disclosure;

FIG. 7 a front view of the optical elements provided within a distal imaging attachment cap of the exemplary imaging system/apparatus of FIG. 1;

FIG. 8 a side view of the imaging system/apparatus, optical elements and distal imaging attachment cap, as shown in FIG. 7;

FIG. 9 is a set of illustrations providing external distal image attachments and an overlapping field external display Diagram according to exemplary embodiments of the present disclosure;

FIG. 10 is a set of exemplary images providing exemplary testing results achieved using the exemplary system, method and/or computer-accessible medium according to the exemplary embodiments of the present disclosure;

FIG. 11 is a side cross-sectional block diagram of an imaging cap of a system/apparatus according to yet another exemplary embodiment of the present disclosure;

FIG. 12 is a perspective view of side/backward view filters and housing according to an exemplary embodiment of the present disclosure;

FIGS. 13(a) and 13(b) are side and front views, respectively, of forward view filters and housing according to another exemplary embodiment of the present disclosure;

FIGS. 14(a) and 14(b) are side and front views, respectively, of beam splitters and housing according to still another exemplary embodiment of the present disclosure;

FIGS. 15(a)-15(c) are side, side perspective and front perspective views, respectively, of forward view filter housing and beam splitter housing according to yet another exemplary embodiment of the present disclosure; and

FIGS. 16(a) and 16(b) are side perspective and front perspective views of side/backward view filter housing, forward view filter housing and beam splitter housing according to yet another exemplary embodiment of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures, and/or the appended claims.

### DETAILED DESCRIPTION OF EXEMPLARY EMBODIMENTS

Using the exemplary embodiments of the apparatus, system and method of the present disclosure, it is possible to facilitate a visualization of a plurality of fields of view, e.g., at a plurality of angles with respect to the long axis of the endoscope by multiplexing image fields of view using an optical apparatus. In one exemplary embodiment of the present disclosure, an optical apparatus/system can be provided which can be partially reflective and/or may be a polarization or wavelength selective such that certain wavelengths or polarization states are directed to and/or received from different field angles and therefore illuminate and/or receive different fields of view.

The exemplary states may be altered by changing the characteristics of the optics or the optical characteristics of the light, such as the wavelengths or the polarization state. For example, such changes of wavelengths can be different bands of wavelengths in the RGB spectrum. Alternatively, the different wavelengths may be comprised of different wavelength bands in the visible and NIR spectrum. Furthermore, e.g., the characteristic(s) of the light is not changed by the optical apparatus, but the images are separated using software algorithms. In yet another embodiment, the optical apparatus contains a beam splitter. In a further exemplary embodiment of the present disclosure, the optical apparatus can be configured and/or structured to be within a cap that can be attached to the distal end of an endoscope, a catheter, a borescope, and/or a laparoscope device. For example, the cap can be disposable, and/or can contain one or more apertures or openings to allow the passage of devices, fluids, or tissue to effect a change in the anatomic structure.

According to another exemplary embodiment of the present disclosure, the arrangement of optical elements coupled with or to certain endoscopes, and exemplary signal processing methods can facilitate an acquisition of continuous multi-directional views, without the need for additional auxiliary imaging devices deployed through the endoscope accessory channel.

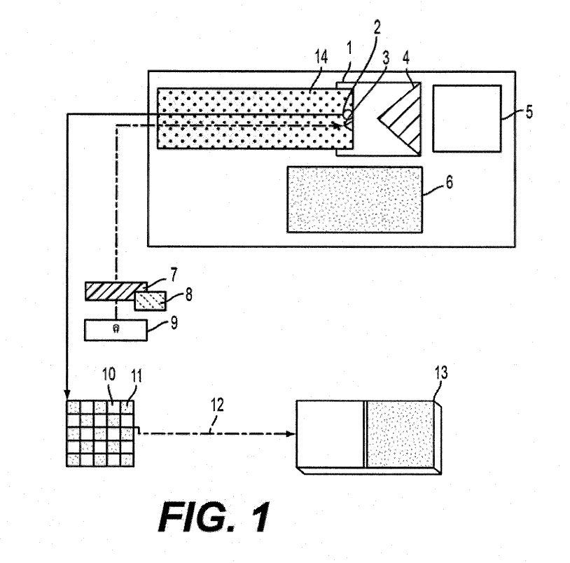

FIG. 1 shows a side cross-sectional block diagram of an imaging system/apparatus and optical elements thereof according to an exemplary embodiment of the present disclosure. For example, a distal imaging attachment cap 1 of the exemplary imaging system of FIG. 1 can be facilitated in an endoscope 14. The attachment cap 1 can contain an optical element/arrangement 4 which can include certain multiple configurations, such as but not limited to a fiber optic bundle, a tapered fiber optic bundle, a cone mirror, a partial cone mirror, a pentagon mirror, an inverted pyramid mirror, a prism, and/or multiple mobile optical elements. The use of such exemplary optical element/arrangement 4 can achieve, e.g., a side and retrograde endoscopic view while maintaining the endoscope's field of view 5, such as the forward field of view. The exemplary optical element 4 may also have or applied thereto a customized reflective material to facilitate a detailed and customized manipulation of the field of view or wavelengths. Such exemplary arrangement can facilitate the user of the exemplary endoscopic system to view both the forward field of view 5 and fields of view located to the side and retrograde 6 to the endoscope's objective lens 2 and endoscope light 3.

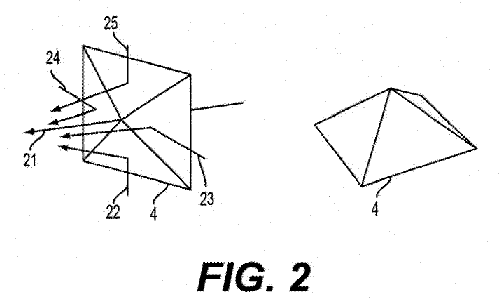

According to another exemplary embodiment of the present disclosure, as shown in FIG. 2, the exemplary optical element 4 can be, e.g., a 4-faceted pyramid dichroic mirror which can transmit and/or reflect radiations (e.g., electromagnetic radiations, including light, etc.) with different characteristics. For example, the exemplary characteristics can include and/or be wavelengths or polarizations. The first and second radiations can have spectral regions in red, green and blue bands which likely do not substantially overlap with one another (at least for the most part). In addition or alternatively, the first and second radiations can have a specific polarization status. For example, the first radiation can be directed in a forward direction 21, and the second radiation can be directed in a backward direction or side directions (e.g., directions 22, 23, 24, 25).

Further, turning to FIG. 1, according to an exemplary embodiment of the present disclosure, it is possible to facilitate a toggling via a manual and/or electronic switch 8 (which can include a modulation arrangement), e.g., to apply an exemplary procedure to filter, polarize, bend and/or exclude predetermined wavelength(s) of one or more radiations (e.g., lights) 7 of an endoscopic light/radiation source 9. As indicated herein, the distal imaging attachment cap 1 can be placed at the distal tip of the endoscope 14.

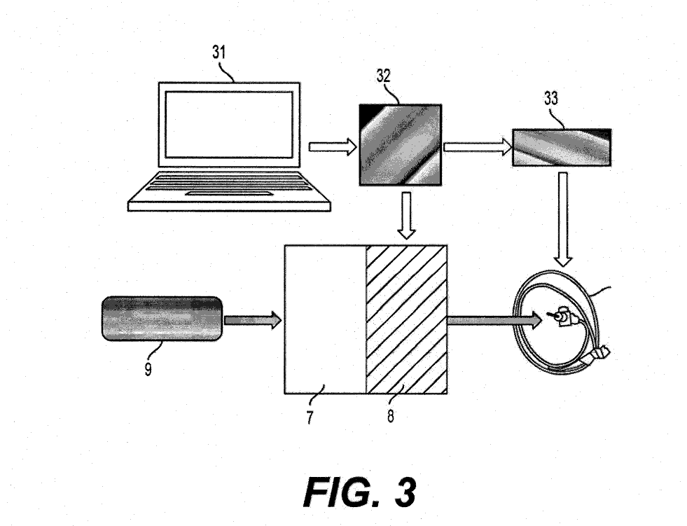

According to another exemplary embodiment of the present disclosure, as shown in FIG. 3, a system can be provided (which can include but not limited to one or more of, e.g., computer 31, video capture device and synchronization signal generator 32, and endoscope video processor 33). The endoscopic arrangement 14 can be associated with the radiation source arrangement (which can include but not limited to one or more of, e.g., endoscopic light/radiation source 9, an exemplary procedure to filter, polarize, bend and/or exclude predetermined wavelength(s) of the radiation(s) 7, and manual and/or electronic switch 8) and a detector arrangement. Further, as indicated herein above, the radiation source arrangement can include the modulation arrangement (including, e.g., element 8) which can be configured to modulate the first and second radiations. The computer 31 and/or the signal generator 32 can be configured to synchronize the radiation source arrangement (including, e.g., elements 8, 9) and/or the entire system (including e.g., elements 31, 32, and 33). As an alternative or in addition, the computer 31 and/or the signal generator 32 can be configured to (i) synchronize the modulation arrangement (including, e.g., element 8) and the detector arrangement, and/or (ii) control the system to detect signals from the anatomical structure(s) illuminated by the first and second radiation, and separate the signals based the synchronization with the modulation arrangement (e.g., element 8).

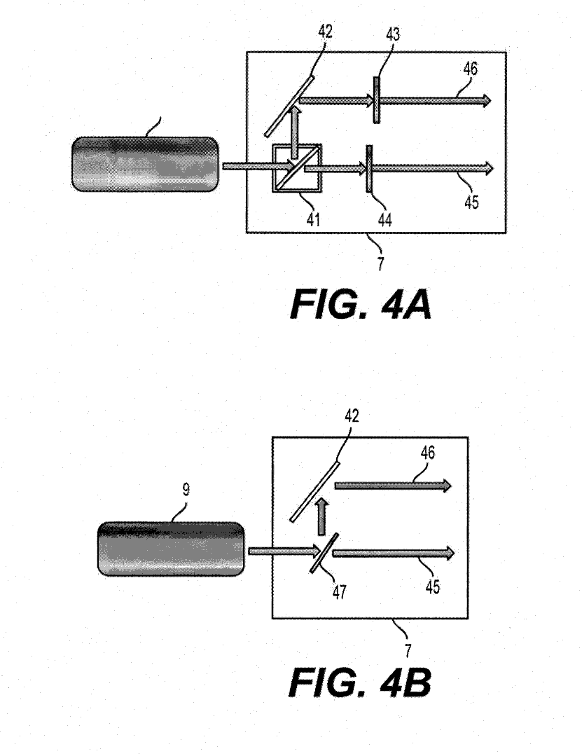

According to yet another exemplary embodiment of the present disclosure, as shown in FIG. 4(a), an exemplary modulation arrangement of another exemplary embodiment of the present disclosure can include a beam splitter 41 to divide the radiation (e.g., light and/or beam) into two beam paths. For example, one beam can pass a filter for predetermined wavelength(s) or polarization(s) 44 to provide the first radiation 45. The other beam can be reflected by a mirror 42, and can pass another filter for another predetermined wavelength(s) or polarization 43 with different characteristics compared with the wavelength(s) or polarization 44 to provide the second radiation 46.

Further, as shown in FIG. 4(b), another exemplary modulation arrangement according to still another exemplary embodiment of the present disclosure can include a beam splitter for predetermined wavelength(s) or polarization 47 to provide the first radiation 45 and the second radiation 46.

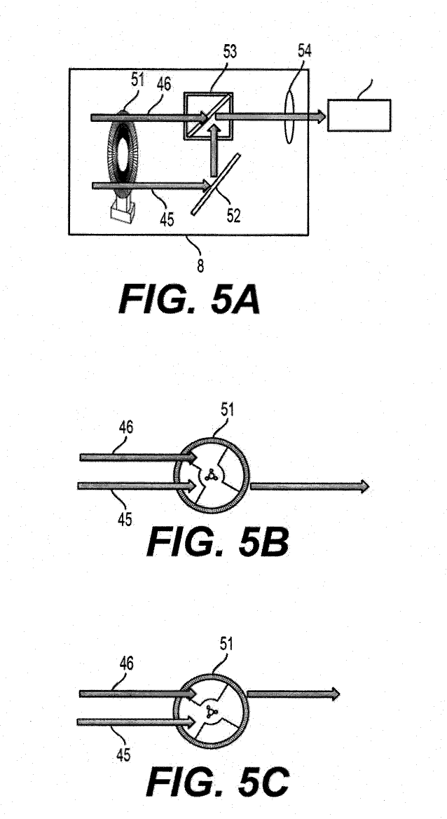

FIGS. 5(a)-5(c) illustrate block diagrams of various exemplary electronic switches according to further exemplary embodiments of the present disclosure. The exemplary electronic switches of FIGS. 5(a)-5(c) can include an optical chopper 51 synchronized with the computer 31 and/or the signal generator 32 (shown in FIG. 3). The first radiation 45 and second radiation 46 can be alternatively coupled into the endoscope 14 by exemplary optical components (e.g., a mirror 52, a beam splitter 53, and a lens 54). Such exemplary optical components can be switched by the optical chopper's positions, as shown in FIGS. 5(b) and 5(c).

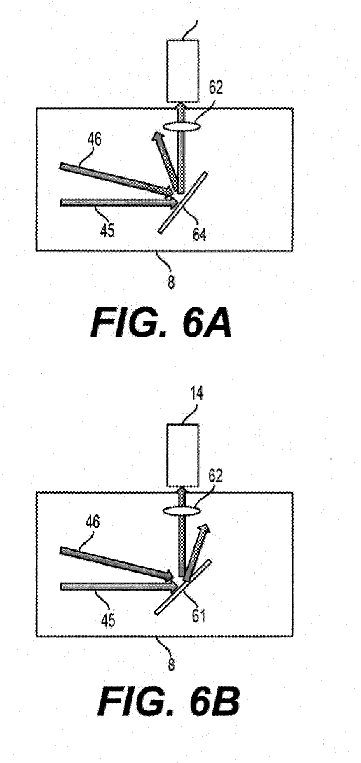

FIGS. 6(a) and 6(b) show another exemplary electronic switch arrangement according to yet another exemplary embodiment of the present disclosure, provided in different switch position. The exemplary switch arrangement of FIGS. 6(a) and 6(b) can include a galvo scanner 61 which can be synchronized with the computer 31 and/or the signal generator 32 (shown in FIG. 3). For example, the first radiation 45 and the second radiation 46 can be alternatively coupled into the endoscope 14 by exemplary optical components (e.g., lens 62) switched by the galvo scanner's positions as shown in FIGS. 6(a) and 6(b).

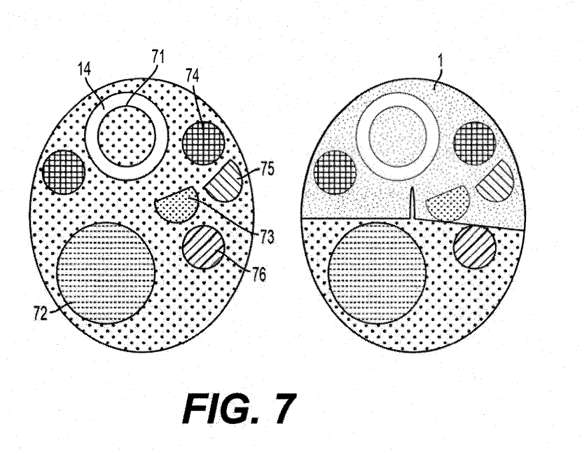

According to another exemplary embodiment of the present disclosure, as shown in FIG. 7, the exemplary distal imaging attachment cap 1 can facilitate a use of a fluid delivery channel 76 and/or an accessory channel 72 to maintain its original use by providing a non-obstructive pathway for an endoscopic manipulation within the endoscope 14 via the accessory channel 72.

### Exemplary Image Processing

In one exemplary embodiment of the present disclosure, with reference to FIG. 1, the exemplary system/apparatus/method can be used for a simultaneous or controlled switching between the above described forward field of view 5 and the side/retrograde field of view 6. In order to facilitate accurate localization of target lesions obtained with the exemplary imaging system, an exemplary procedure 12 (which can be used to program a processing hardware arrangement, such as, e.g., a computer) can be used to deconstruct a wavelength/polarization “profile” of each field of view 10, 11 by electronically splitting native and multidirectional fields of view.

Using an a light/radiation source of the endoscope 14, exemplary selective filtering of, e.g., white light to facilitate only the reflectance or transmission phase to be analyzed can be accomplished by placing applying a filter at the endoscope's connection to its processing arrangement (e.g., the processor). Toggling between the on and off phases, e.g., manually (such as with a manual foot pedal), automatically or via an electronic switch, the reflected or transmitted light/radiation can then be deconstructed via a further procedure which can program or configure the processing arrangement to continuously display the forward and multidirectional fields of view 13.

According to yet further exemplary embodiment of the present disclosure, another procedure can be provided which can program or configure the processing arrangement to deconstruct each pixel, and display the two profiles determined by the reflective transmission wavelengths, polarizations or characteristic properties established by a special arrangement 7, the optical element(s) 4 and angles of observation of each field of view 5, 6.

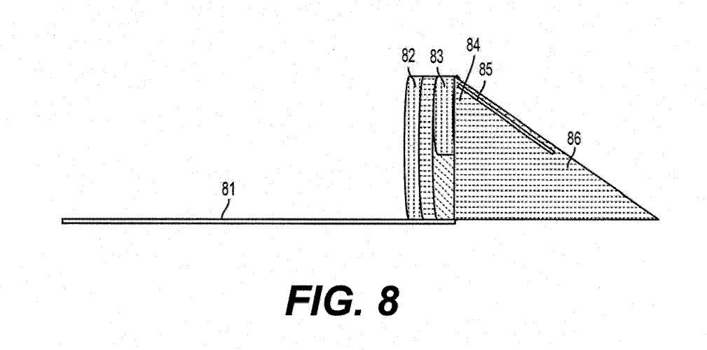

The exemplary imaging system of FIG. 1 can also use of an alternative light source which can be deployed, e.g., via the cap irrigation channel 81 (shown in FIG. 8). The use of such light source via the irrigation channel 81 can provide and/or facilitate, e.g., a further selective manipulation of the reflectance and transmission frequencies for an improved discretion between the phases for an exemplary image manipulation via a procedure which can program or configure the processing arrangement to perform such exemplary function.

### Exemplary Application of Exemplary Embodiment

### Exemplary Cap Design

According to one exemplary embodiment of the present disclosure, a plastic, transparent, semi-flexible disposable cap 1 can be fitted over the distal tip of the endoscope 14 via a friction fit configuration 82, as shown in FIG. 8. The exemplary design and/or configuration of this cap 1 can be provided in various ways, e.g., depending on the indication of the exemplary endoscopic procedure. Shapes of the exemplary cap 1 can include, but are not limited to oblique or perpendicular angled shapes, in respect to the distal aspect of the endoscope 14 and a location of the objective lens 2.

In a further exemplary embodiment of the present disclosure, the distal imaging attachment is designed to be in a specific orientation so as to facilitate the native functions of the endoscope to continue to operate without an interruption. To facilitate the function of, e.g., cleaning the endoscopes objective lens 71, a light guide 74, an air nozzle 73, and a water nozzle 75 (as shown in FIG. 7), the exemplary cap 1 can include a clearance chamber 83 (as shown in FIG. 8), which can seal the distal apparatus away from luminal liquid and contents, while continuing to facilitate the instillation of water for imaging and cleaning. This above described exemplary clearance chamber 83 can contain a perforation located above the accessory chamber 86 to facilitate suctioning of contents of the clearance chamber 83. To facilitate the distal imaging cap to be cleaned, a water jet output channel (e.g., the fluid delivery chamber) 76 can be provided which is structured and/or designed to be unobstructed by the exemplary cap 1. Furthermore, to provide more aggressive cleansing, e.g., the distal imaging cap is also structured and/or designed with an irrigator port 81 which can facilitate the attachment of a lavage device or syringe to aid in a clearance of liquid and/or debris from the distal attachment cap 1.

Further, the exemplary cap 1 can be coupled with multiple optical elements 85 in the optical chamber 84.

### Overlapping Field Cap Design

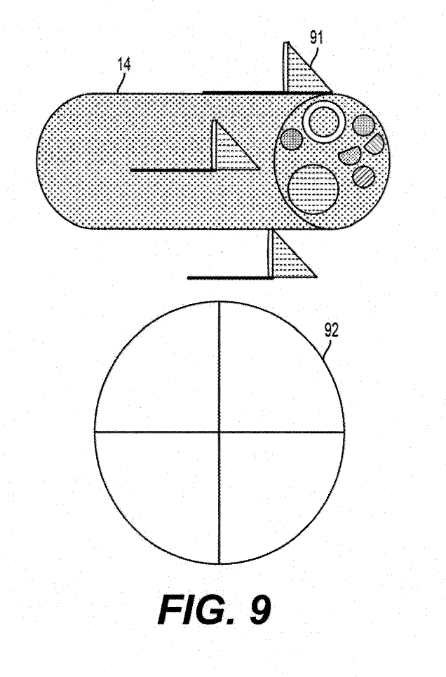

According to yet another exemplary embodiment of the present disclosure, it is possible to use a plastic, transparent, semi-flexible and disposable cap, which can facilitate a circular configuration and arrangement of multiple imaging detectors within a small collar 91, as shown in FIG. 9. This exemplary collar 91 can facilitate overlapping, multidirectional and circumferential views of the desired target sample (e.g., organ) or space being inspected. This exemplary configuration can facilitate the use of multiple light sources and independent optical sensors, e.g., bypassing a preference to alter the conventional endoscopes light source. Exemplary image processing of images obtained using the system, apparatus and method according to the present disclosure can be accomplished using the exemplary procedures implemented on the exemplary processing arrangement, as described herein. For example, depending on the number of optical elements placed within the exemplary imaging collar-cap design, an exemplary procedure implemented on the exemplary processing arrangement according to an exemplary embodiment of the present disclosure can assist in an alignment of the signals to provide, e.g., a 360 degree, multidirectional field of view 92, as shown in FIG. 9.

### Exemplary Testing

Further exemplary testing was performed using the following: (1) Polka dot beam splitter, (2) 50:50 beam splitter AOI 45 degree, (3) long pass dichroic mirror, 50% Trans. Refl. At 567 nm, (4) cone mirror or (5) 395/495/610 nm Triple-edge dichroic beam splitter installed at multiple distances distal to the endoscopes objective leans. Preliminary testing with both a white light and infrared light source was successful in demonstrating that selective observation of the forward and retrograde views could be accomplished if the optical element was oriented at an angle such as a 45 degree, 30 degree, or 60 degree angle to the endoscopes objective lens.

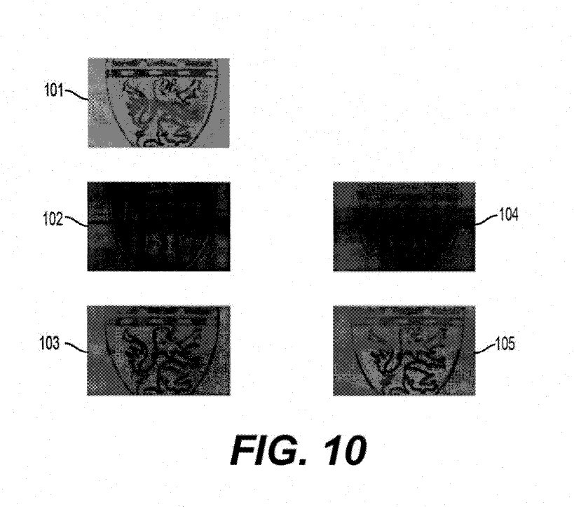

FIG. 10 shows a set of exemplary images achieved using the exemplary system, method and/or computer-accessible medium according to the exemplary embodiments of the present disclosure. Such exemplary images were based on exemplary testing result using an exemplary software separation via a simultaneously illumination and utilizing a 442/505/635 nm Yokogawa dichroic beamsplitter installed at the distal to a CCD camera with lens. Massachusetts General Hospital (“MGH”) logo and Harvard Medical School (“HMS”) logo were used as the image targets, placed in front of, and at side of the dichroic beamsplitter, respectively. In this testing, the white light source was not modulated and illuminated on the two image targets simultaneously. The separately captured exemplary individual images of the logos are shown in FIG. 10 as MGH 102, and HMS 103. A captured exemplary combined image 101 with the two logos in positions at the same time was the image intended to be processed. The exemplary procedure 12 described herein above with respect to FIG. 1 (which may be used to program a processing hardware arrangement, such as, e.g., a computer) can be utilized to deconstruct the wavelength “profile” of each field of view by splitting the native and multidirectional fields of view with the two logos. For example, the exemplary procedure 12 can be one or more programs including, but not limited to, e.g., Neural Network and/or Independent Component Analysis, or other procedure/program which can configure the processing hardware arrangement to separate the two views from the captured combined image 101. The exemplary reconstructed images of each field of view are shown in FIG. 10 as images 104, 105, respectively. The exemplary software based separation, which splits the two views as described herein, can significantly reduce the complexity of various components/parts of the procedure, system and computer-accessible medium according to the exemplary embodiments of the present disclosure.

An exemplary integration of such exemplary configuration that is associated with a distal imaging cap which is is coupled with various optical elements has been described herein.

According to a further exemplary embodiment of the present disclosure, e.g., the characteristics of the light may not be changed by an optical apparatus, and instead, the images can be separated using software processing procedures.

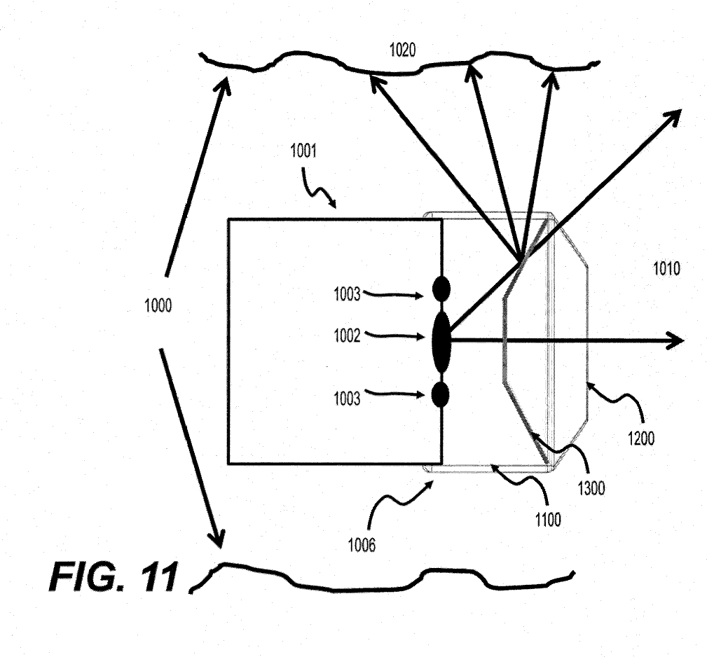

FIG. 11 shows a side cross-sectional block diagram of an imaging system/apparatus and optical elements thereof according to a further exemplary embodiment of the present disclosure, provided within a lumen 1000. For example, a distal imaging attachment cap 1006 (which can include side/backward view filter arrangement/housing 1100, forward view filter arrangement/housing 1200, and beam splitter arrangement/housing 1300) of the exemplary imaging system illustrated in FIG. 11 can be facilitated or otherwise provided in an endoscope 1001. The attachment cap 1006 can include such arrangements/housings 1100, 1200, 1300 or other optical arrangements which can have certain multiple configurations, such as but not limited to, a cone mirror, a partial cone mirror, a pentagon mirror, an inverted pyramid mirror, a prism, and/or multiple mobile optical elements. The exemplary use of such exemplary optical element/arrangements/housings 1100, 1200, 1300 can achieve, e.g., a side and retrograde endoscopic view 1020, while maintaining endoscope's forward field of view 1010, such as, e.g., the forward field of view. The exemplary optical elements/arrangements/configurations 1100, 1200, 1300 can also have or applied thereto a customized reflective material to facilitate a detailed and/or a customized manipulation of the field of view or wavelengths. Such exemplary arrangement/configuration can facilitate the user of the exemplary endoscopic system to view both the forward field of view 1010 and fields of view located to the side and retrograde 1020 to the endoscope's objective lens 1002 and an endoscope light arrangement 1003.

In particular, referring to FIG. 11, the light (or other electro-magnetic radiation) provided from the endoscope light arrangement 1003 can be partially reflected by the beam splitter arrangement/housing 1300, and transmitted through the side/backward view filter arrangement(s)/housing(s) 1100. The scattered side/backward light from the tissue can be provided back to the endoscope objective lens 1002 to facilitate the side and retrograde view 1020. The light from the endoscope light arrangement 1003 can be partially transmitted through the beam splitter arrangement/housing 1300, and transmitted through the forward view filter arrangement(s)/housing(s) 1200. The scattered forward light from the tissue can be provided back to the endoscope objective lens 1002 to provide the forward field of view 1010.

Further, according to an exemplary embodiment of the present disclosure, it is possible to facilitate a toggling via an electronic and/or manual switch, e.g., to apply a procedure to filter, and/or exclude predetermined wavelength(s) of lights provided from the endoscope light arrangement 003 of an endoscopic light/radiation source. According to a further exemplary embodiment of the present disclosure, the exemplary switch can be or include a scanner to scan the light source beam to different filter sets, synchronized by the control system, such as, e.g., a programmed computer. In another exemplary embodiment of the present disclosure, the exemplary switch can be or include an optical chopper which is configured to block one or more of particular paths of the light source beam to or from different filter sets, e.g., synchronized by the control system, such as the programmed computer.

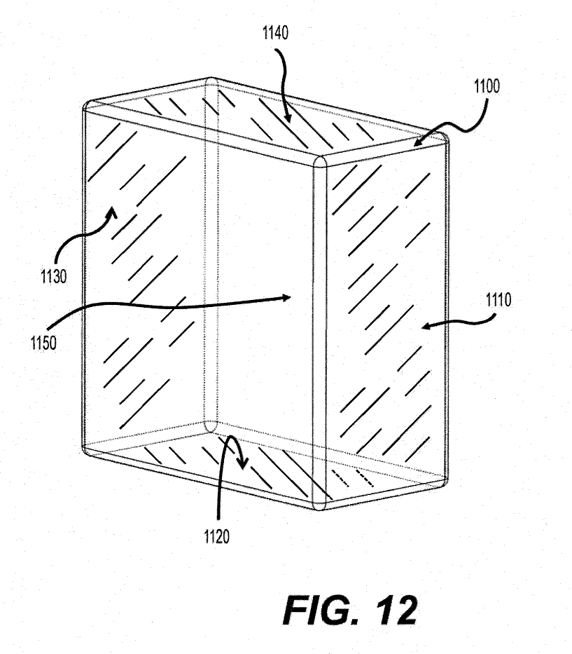

According to still exemplary embodiment of the present disclosure, as shown in FIG. 12 illustrating a perspective view of side/backward view filters and housing, the imaging device can have optical filter arrangement(s)/housing(s) 1100 at all facets or parts of the facets (e.g., a side view direction). According to another exemplary embodiment of the present disclosure, the number of facets at the side/backward views can be at least four facets which can also be filters 1110, 1120, 1130, 1140, as shown in the FIG. 12, or any other number of facets. Further, such filters/facets 1110, 1120, 1130, 1140 can be provided around or about a hollow internal portion 1150. The optical filters and/or windows 1110, 1120, 1130, 1140 can be designed, structured and/or configured for a transmission of one or more predetermined wavelength(s), such as different bands of wavelengths in the RGB spectrum. According to another exemplary embodiment of the present disclosure, the optical filters and/or windows 1110, 1120, 1130, 1140 can be provided at or in different tilted angles.

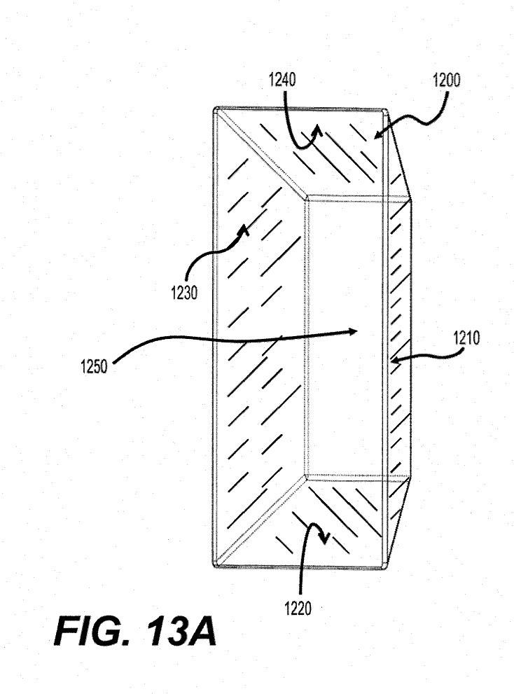

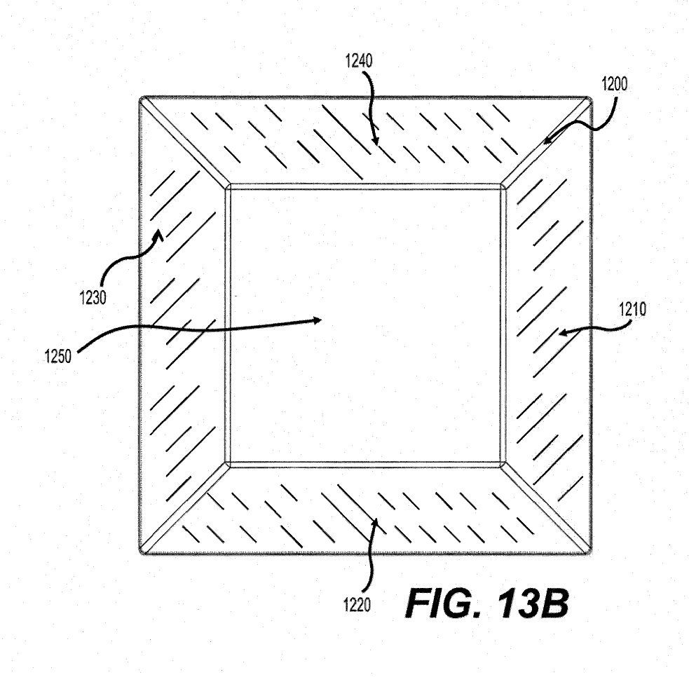

In yet another exemplary embodiment of the present disclosure, as shown in FIGS. 13(a) and 13(b), exemplary optical filter(s) and/or housing(s) 1200 can be provided at other facets (e.g., a forward view direction) of the exemplary imaging attachment cap. According to a further exemplary embodiment of the present disclosure, the number of facets at the forward views can be at least four facets which can also be filters 1210, 1220, 1230, 1240, as shown in the FIGS. 13(a) and 13(b), or any other number of facets. Further, such filters/facets 1210, 1220, 1230, 1240 can be provided around or about a hollow internal portion 1250. The optical filters and/or windows 1210, 1220, 1230, 1240 can also be designed, structured and/or configured for a transmission of predetermined wavelength(s), such as, e.g., different bands of wavelengths in the RGB spectrum. According to another exemplary embodiment of the present disclosure, the optical filters and/or windows 1210, 1220, 1230, 1240 can be placed, provided or put at or in different tilted angles.

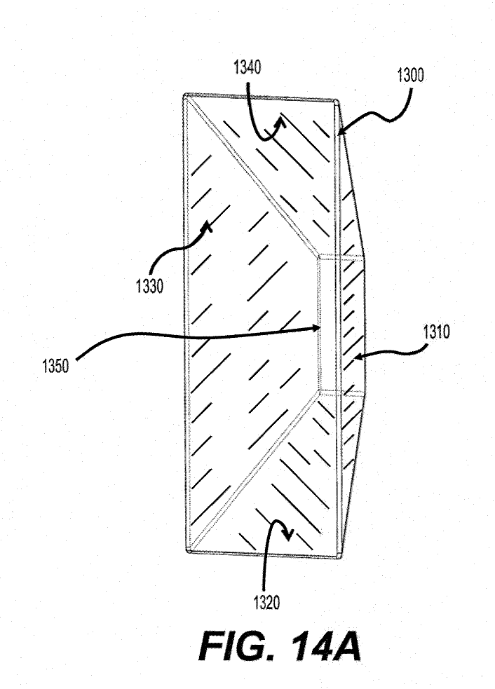

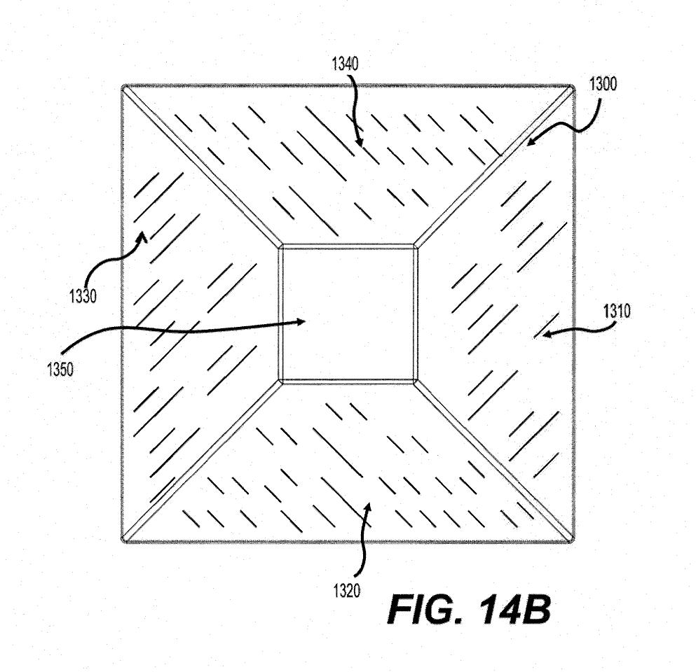

According to yet a further exemplary embodiment of the present disclosure, as shown in FIGS. 14(a) and 14(b), the exemplary beam splitter and/or housing 1300 can be provided in front of the endoscope in the exemplary imaging attachment cap. In one example, the number of facets at the forward views can be at least four facets which can also be filters 1310, 1320, 1330, 1340, as shown in the FIGS. 14(a) and 14(b), or any other number of facets. Further, such filters/facets 1310, 1320, 1330, 1340 can be provided around or about a hollow internal portion 1350. In still another exemplary embodiment of the present disclosure, a 50/50 beam splitter can be used. Further, e.g., other optical component(s) can be used to split the light, such as a dichroic mirror. According to yet further exemplary embodiment of the present disclosure, the surface of the beam splitter can be a cone shape.

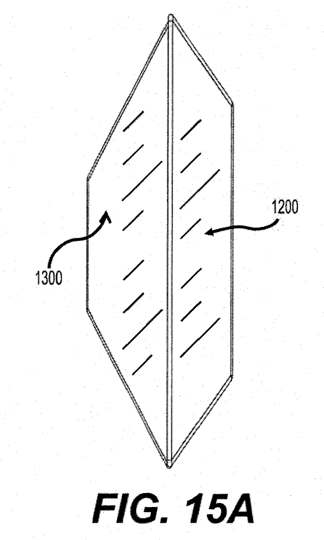

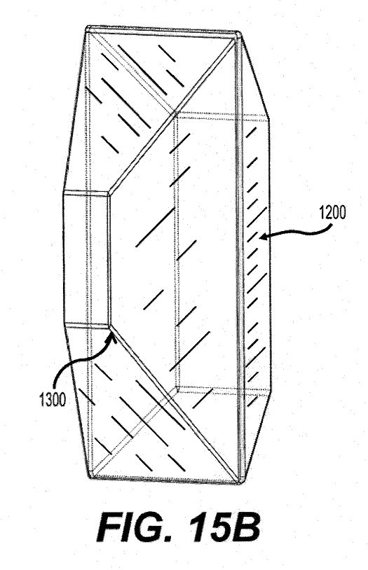

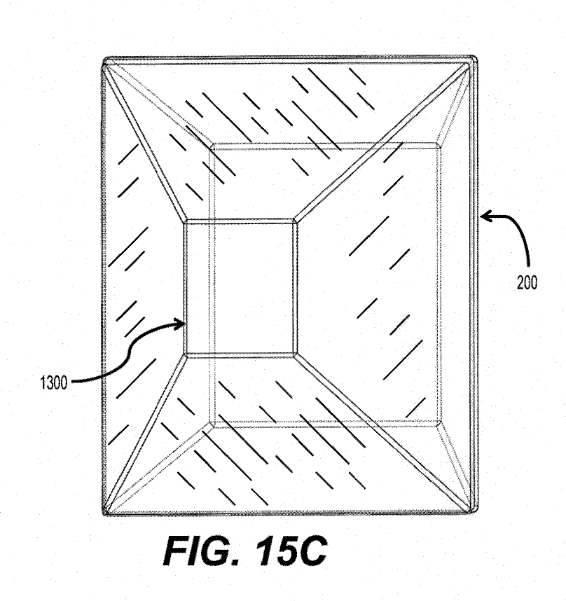

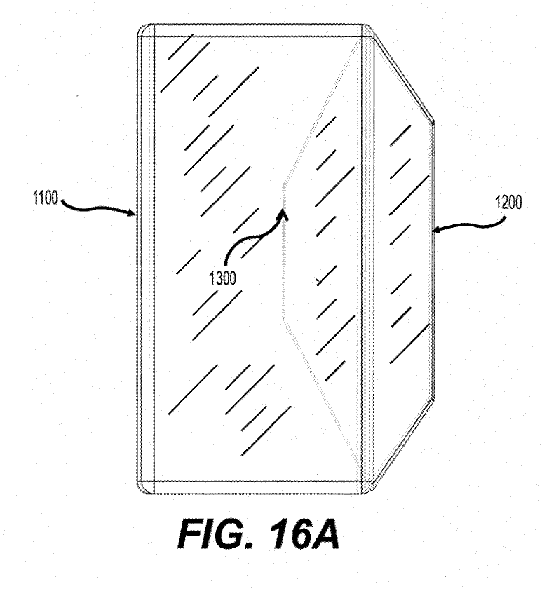

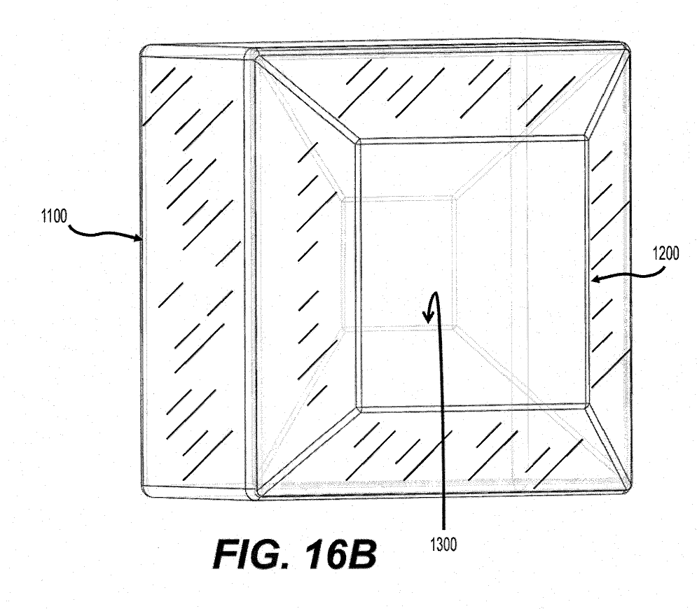

In an additional exemplary embodiment of the present disclosure, as shown in FIGS. 15(a)-16(b), the relative surface and position of and/or in the side/backward view filter arrangement/housing 1100, the forward view filter arrangement/housing 1200, and the beam splitter arrangement/housing 1300 can be changed according to different requirements such as the view angles.

According to another exemplary embodiment of the present disclosure, as shown in FIGS. 12-16(b), the exemplary distal imaging attachment cap, which can include respective hollow internal portions 1150 (see FIG. 12), 1250 (see FIGS. 13(a) and 13(b)), 1350 (see FIGS. 14(a) and 14(b)), can facilitate an exemplary use of a fluid delivery channel and/or an accessory channel of an endoscope to maintain its original use by providing a non-obstructive pathway for an endoscopic manipulation within the endoscope via the accessory channel.

In addition, according to a further exemplary embodiment of the present disclosure, a procedure (e.g., in a form of a software computer program) can be provided which can program and/or configure the processing arrangement to deconstruct each pixel, and display the two profiles determined by the reflective, and transmissive wavelengths established by wavelength filters, the optical element(s)/housing(s) 1100, 1200, and 1300, and angles of observation of each field of view 1010, 1020.

In yet an additional exemplary embodiment of the present disclosure, algorithm, procedure(s) and/or software program(s) can be provided to program or otherwise configure a computer to correct any distortion of the anatomical structure under examination induced by the exemplary imaging apparatus, reconstruct the relative positions of different views related to the forward view of the endoscope, and/or balance the color. It is also possible to facilitate and/or provide a toggling procedure to selectively display any individual field of views via a manual and/or electronic switch, e.g., using the algorithms and/or computer software by programming the computer to perform the same.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with any OCT system, OFDI system, SD-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

### EXEMPLARY REFERENCES

1. Winawer S J, Zauber A G, Ho M N, O'Brien M J, Gottlieb L S, Sternberg S S, Waye J D, Schapiro M, Bond J H, Panish J F, et al. Prevention of colorectal cancer by colonoscopic polypectomy. The National Polyp Study Workgroup. N Engl J Med 1993;329:1977-81.
2. Bressler B, Paszat L F, Vinden C, Li C, He J, Rabeneck L. Colonoscopic miss rates for right-sided colon cancer: a population-based analysis. Gastroenterology 2004;127:452-6.
3. Rex D K, Cutler C S, Lemmel G T, Rahmani E Y, Clark D W, Helper D J, Lehman G A, Mark D G. Colonoscopic miss rates of adenomas determined by back-to-back colonoscopies. Gastroenterology 1997;112:24-8.
4. Haseman J H, Lemmel G T, Rahmani E Y, Rex D K. Failure of colonoscopy to detect colorectal cancer: evaluation of 47 cases in 20 hospitals. Gastrointest Endosc 1997;45:451-5.
5. Pellise M, Fernandez-Esparrach G, Cardenas A, Sendino O, Ricart E, Vaquero E, Gimeno-Garcia A Z, de Miguel C R, Zabalza M, Gines A, Pique J M, Llach J, Castells A. Impact of wide-angle, high-definition endoscopy in the diagnosis of colorectal neoplasia: a randomized controlled trial. Gastroenterology 2008;135:1062-8.
6. Rex D K, Chadalawada V, Helper D J. Wide angle colonoscopy with a prototype instrument: impact on miss rates and efficiency as determined by back-to-back colonoscopies. Am J Gastroenterol 2003;98:2000-5.
7. Deenadayalu V P, Chadalawada V, Rex DK. 170 degrees wide-angle colonoscope: effect on efficiency and miss rates. Am J Gastroenterol 2004;99:2138-42.
8. Matsushita M, Hajiro K, Okazaki K, Takakuwa H, Tominaga M. Efficacy of total colonoscopy with a transparent cap in comparison with colonoscopy without the cap. Endoscopy 1998;30:444-7.
9. Lee Y T, Hui A J, Wong VW, Hung L C, Sung J J. Improved colonoscopy success rate with a distally attached mucosectomy cap. Endoscopy 2006;38:739-42.
10. Kondo S, Yamaji Y, Watabe H, Yamada A, Sugimoto T, Ohta M, Ogura K, Okamoto M, Yoshida H, Kawabe T, Omata M. A randomized controlled trial evaluating the usefulness of a transparent hood attached to the tip of the colonoscope. Am J Gastroenterol 2007;102:75-81.
11. Harada Y, Hirasawa D, Fujita N, Noda Y, Kobayashi G, Ishida K, Yonechi M, Ito K, Suzuki T, Sugawara T, Horaguchi J, Takasawa O, Obana T, Oohira T, Onochi K, Kanno Y, Kuroha M, Iwai W. Impact of a transparent hood on the performance of total colonoscopy: a randomized controlled trial. Gastrointest Endosc 2009;69:637-44.
12. Horiuchi A, Nakayama Y. Improved colorectal adenoma detection with a transparent retractable extension device. Am J Gastroenterol 2008;103:341-5.
13. Hewett D G, Rex D K. Cap-fitted colonoscopy: a randomized, tandem colonoscopy study of adenoma miss rates. Gastrointest Endosc 2010;72:775-81.
14. Triadafilopoulos G, Watts H D, Higgins J, Van Dam J. A novel retrograde-viewing auxiliary imaging device (Third Eye Retroscope) improves the detection of simulated polyps in anatomic models of the colon. Gastrointest Endosc 2007;65:139-44.
15. Leufkens A M, DeMarco D C, Rastogi A, Akerman P A, Azzouzi K, Rothstein R I, Vleggaar F P, Repici A, Rando G, Okolo P I, Dewit O, Ignjatovic A, Odstrcil E, East J, Deprez P H, Saunders B P, Kalloo A N, Creel B, Singh V, Lennon A M, Siersema P D. Effect of a retrograde-viewing device on adenoma detection rate during colonoscopy: the TERRACE study. Gastrointest Endosc 2011;73:480-9.

What is claimed is:

1. An apparatus for imaging at least one anatomical structure, comprising:
a probe imaging first arrangement;
a radiation source second arrangement which provides at least one electro-magnetic radiation; and
a cap third arrangement attached to at least one portion of the endoscopic arrangement, and containing an optical arrangement which, upon impact by the at least one electro-magnetic radiation and based thereon, transmits a first radiation and reflects a second radiation, wherein the first radiation impacts at least one first portion of the at least one anatomical structure, and the second radiation impacts at least one second portion of the at least one anatomical structure, the first and second portions being at least partially different from one another,
wherein the first and second radiations have characteristics which are different from one another, and
wherein the third arrangement includes a hollow internal portion that facilitates an unobstructed passage of at least one of fluid or instrument within an accessory channel of the first arrangement.

2. The apparatus according to paragraph 1, wherein the characteristics are wavelengths or polarizations.

3. The apparatus according to paragraph 1, further comprising a detector arrangement, wherein the probe imaging first arrangement is associated with the radiation source arrangement and the detector arrangement.

4. The apparatus according to paragraph 1, wherein the first and second radiations have spectral regions in red, green and blue band which do not substantially overlap with one another.

5. The apparatus according to paragraph 1, wherein the first radiation is directed in a forward direction, and wherein the second radiation is directed in a backward direction or a side direction.

6. The apparatus according to paragraph 5, wherein the second radiation simultaneously illuminates between 270 and 360 degrees of a field of view.

7. The apparatus according to paragraph 3, wherein the radiation source second arrangement includes a modulation arrangement which is configured to modulate the first and second radiations.

8. The apparatus according to paragraph 7, further comprising an electronic arrangement which is configured to synchronize the second arrangement and the detector arrangement.

9. The apparatus according to paragraph 8, further comprising an electronic arrangement which is configured (i) synchronize the modulation arrangement and the detector arrangement, and (ii) control the detector arrangement to detect signals from the at least one anatomical structure illuminated by the first and second radiations, and separate the signals based the synchronization with the modulation arrangement.

10. The apparatus according to paragraph 1, wherein the at least one anatomical structure is a luminal anatomical structure.

11. The apparatus according to paragraph 1, wherein the third arrangement includes at least one opening which facilitates a passage of instrumentation, air gasses and fluids therethrough.

12. The apparatus according to paragraph 1, wherein the first and second radiations have a specific polarization status.

13. The apparatus according to paragraph 6, wherein the cap includes an optical structure, which, upon impact by the at least one electro-magnetic radiation and based thereon, cause a resultant radiation to be at least side, retrograde and forward directions of the probe imaging first arrangement with respect to a viewing direction thereof.
